(19) United States
(12) Patent Application Publication
(10) Pub. No.: US 2024/0226197 A1
(43) Pub. Date: Jul. 11, 2024

(54) METHODS OF TREATING DISEASES AND CONDITIONS, SUCH AS INFLAMMATORY DISEASES, CANCER, CARDIOVASCULAR DISEASE, DIABETES AND OBESITY WITH THE PROBIOTIC LACTOBACILLUS JOHNSONII 456

(71) Applicant: Robert H. Schiestl, Van Nuys, CA (US)

(72) Inventor: Robert H. Schiestl, Van Nuys, CA (US)

(21) Appl. No.: 17/858,364

(22) Filed: Jul. 6, 2022

(51) Int. Cl. A61K 35/747; A61P 1/00; A61P 3/10; A61P 9/10; A61P 11/00; A61P 35/00

(52) U.S. Cl. CPC A61K 35/747; A61P 1/00; A61P 3/10; A61P 9/10; A61P 11/00; A61P 35/00

(57) ABSTRACT

Embodiments of the present disclosure recite methods of treating many diseases, such as inflammatory diseases, cancers, cardiovascular diseases, diabetes and obesity or conditions within a patient in need of such treatment by administering Lactobacillus johnsonii 456 to the mammal such as patient or person.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The present disclosure relates generally to methods for treating many diseases, such as inflammatory diseases, namely pneumonia and enteritis by administering a specific probiotic Lactobacillus johnsonii 456 to a mammal, such as an animal or patient or person in need. Pneumonia is an infection that inflames the air sacs in one or both lungs. The air sacs may fill with fluid or pus (purulent material), causing cough with phlegm or pus, fever, chills, and difficulty breathing. A variety of organisms, including bacteria, viruses and fungi, can cause pneumonia. Pneumonia can range in seriousness from mild to life-threatening. It is most serious for infants and young children, people older than age 65, and people with health problems or weakened immune systems. https://www.mayoclinic.org/diseases-conditions/pneumonia/symptoms-causes/syc-20354204

Inflammation of the small intestine is called enteritis. Inflammation (redness and swelling) might be caused by infection, certain medications, radiation treatments, or chronic illnesses such as inflammable bowel disease, ulcerative colitis or Crohn's disease or celiac disease. Symptoms can include pain, diarrhea, vomiting, and blood in the stool. Enteritis can be caused by a range of conditions or medications that affect the small intestine. In some cases, enteritis might be caused by eating or drinking something contaminated by bacteria or viruses. Radiation treatment for cancer can also lead to inflammation in the digestive system. https://www.verywellhealth.com/enteritis-5218257#:˜:text=Inflammation%20of%20the%20small%20intestine,and %20blood%20in %20the%20stool.

Other inflammatory-based diseases that may be treated by the probiotic Lactobacillus johnsonii 456 include: central nervous system conditions (e.g., encephalitis, myelitis),_meningitis (arachnoiditis), peripheral nervous system conditions (neuritis), eye conditions (dacryoadenitis, scleritis, episcleritis, keratitis, retinitis, chorioretinitis, blepharitis, conjunctivitis, uveitis, car conditions (otitis externa, otitis media, labyrinthitis, mastoiditis), cardiovascular system (carditis, endocarditis, myocarditis, pericarditis), vasculitis (arteritis, phlebitis, capillaritis), respiratory system (sinusitis, rhinitis, pharyngitis, laryngitis, tracheitis, bronchitis, bronchiolitis, pneumonitis, pleuritis, mediastinitis), digestive system (stomatitis, gingivitis, gingivostomatitis, glossitis, tonsillitis, sialadenitis/parotitis, cheilitis, pulpitis, gnathitis, esophagitis, gastritis, gastroenteritis, colitis, enterocolitis, duodenitis, ileitis, cacitis, appendicitis, proctitis, hepatitis, ascending cholangitis, cholecystitis, pancreatitis, peritonitis), integumentary system (dermatitis, folliculitis, cellulitis, hidradenitis), musculoskeletal system (arthritis, dematomyositis, myositis, synovids, bursitis, enthesitis, fasciitis, capsulitis, epicondylitis, tendinitis, panniculitis, osteochondritis, chondritis), urinary system (nephritis, ureteritis, cystitis, urethritis), female reproductive systems (oophoritis, salpingitis, endometritis, parametritis, cervicitis, vaginitis, vulvitis, mastitis) male reproductive systems (orchitis, epididymitis, prostatitis, seminal vesiculitis, balanitis, posthitis, balanoposthitis), pregnancy/newborn (chorioamnionitis, funisitis, omphalitis), endocrine system (insulitis, hypophysitis, thyroiditis, parathyroiditis, adrenalitis), lymphatic system (lymphangitis, lymphadenitis). https://www.wikiwand.com/en/lList_of_inflammatory.disorders

The present disclosure also relates generally to methods for treating cancer, such as blood cancer, small intestinal cancer, and/or colon cancer by administering a specific probiotic Lactobacillus johnsonii 456 to a mammal, such as an animal or patient or person in need. Blood cancers affect the production and function of blood cells. Most of these cancers start in the bone marrow where blood is produced. Stem cells in the bone marrow mature and develop into three types of blood cells: red blood cells, white blood cells, or platelets. In most blood cancers, the normal blood cell development process is interrupted by uncontrolled growth of an abnormal type of blood cell. These abnormal blood cells, or cancerous cells, prevent your blood from performing many of its functions, like fighting off infections or preventing serious bleeding.

There are three main types of blood cancers:
Leukemia, a type of cancer found in the blood and bone marrow, is caused by the rapid production of abnormal white blood cells. The high number of abnormal white blood cells are not able to fight infection, and they impair the ability of the bone marrow to produce red blood cells and platelets.
Lymphoma is a type of blood cancer that affects the lymphatic system, which removes excess fluids from your body and produces immune cells.

Lymphocytes are a type of white blood cell that fight infection. Abnormal lymphocytes become lymphoma cells, which multiply and collect in the lymph nodes and other tissues. Over time, these cancerous cells can impair the immune system.
Myeloma is a cancer of the plasma cells. Plasma cells are white blood cells that produce disease- and infection-fighting antibodies in your body. Myeloma cells prevent the normal production of antibodies, leaving the body's immune system weakened and susceptible to infection.
https://www.hematology.org/education/patients/blood-cancers#:˜:text=Blood%20cancers%20affect%20the%20production,white %20blood %20cells%2C%20or%20platelets.

Lymphoma is a blood cancer that affects the lymphatic system, which is responsible for removing excess bodily fluids and producing immune cells. The lymphatic system includes the lymph nodes (lymph glands), spleen, thymus gland and bone marrow. Lymphoma can affect all these areas as well as other organs throughout the body and over time impair the immune system. The primary types of lymphoma are Hodgkin's lymphoma (formerly called Hodgkin's disease) and Non-Hodgkin's lymphoma. Found in the blood and bone marrow, another type of blood cancer is leukemia which results in the overproduction of white blood cells that both inhibits the immune system and impairs the ability of the bone marrow to produce red blood cells and platelets. Myeloma is a cancer of white blood cells found in blood plasma that is responsible for the production of antibodies necessary for the immune system. https://www.mayoclinic.org/diseases-conditions/lymphoma/symptoms-causes/syc-20352638

Colon cancer develops when tumorous growths form in the large intestine. The colon, or large intestine, is where the body draws out water and salt from solid waste. The waste then moves through the rectum and exits the body through the anus. Rectal cancer originates in the rectum, the final several inches of the large intestine, closest to the anus. Colorectal cancer, which describes co-occurring colon cancer and rectal cancer, is common. According to the American Cancer Society (ACS), colorectal cancer is the third most commonly diagnosed cancer in the United States, aside from skin cancers. In 2021, the ACS predicts that 104.270 people in the U.S. will receive a new diagnosis of colon cancer. https://www.medicalnewstoday.com/articles/150496

The present disclosure also relates generally to methods for treating cardiovascular diseases, such as myocardial infarction or heart attack by administering a specific probiotic Lactobacillus johnsonii 456 to a mammal, such as an animal or patient or person in need. Cardiovascular disease (heart disease) refers to a group of diseases that affect the heart and blood vessels of your body, resulting in one or more of the following symptoms: abnormal heart rhythm, heart valve disease, narrowed blood vessels in the heart or other organs caused by plaque, heart contraction issues, and/or heart lining issues. A person may be symptomatic (physically experience the disease) or be asymptomatic (not feel anything at all). https://my.clevelandclinic.org/health/diseases/21493-cardiovascular-disease#:˜:text=Cardiovascular%20diseases%20(CVDs)%20affect%20the,healthcare%2 0provider%20may%20prescribe%20medications.

A myocardial infarction (commonly called a heart attack) is an extremely dangerous condition caused by a lack of blood flow to the heart muscle. The lack of blood flow can occur because of many different factors but is usually related to a blockage in one or more of the heart's arteries. Without blood flow, the affected heart muscle will begin to die, if blood flow isn't restored quickly, a heart attack can cause permanent heart damage and death. New heart attacks happen to about 635,000 people in the United States each year. About 300,000 people a year have a second heart attack. About one in seven deaths in the United States is due to coronary heart disease, which includes heart attacks. https://my.clevelandclinic.org/health/diseases/16818-heart-attack-myocardial-infarction

The present disclosure also relates generally to methods for reducing the glycemic index by administering a specific probiotic Lactobacillus johnsonii 456 to a mammal, such as an animal or patient or person in need.

The present disclosure relates generally to methods for treating diabetes by administering a specific probiotic Lactobacillus johnsonii 456 to a mammal, such as an animal or patient or person in need. Diabetes is caused by high glucose levels in the blood. In the case of type 1 diabetes, the body does not produce insulin, the hormone necessary to break down glucose for cellular metabolism. With type 2 diabetes, the body does not make or use insulin well. https://www.niddk.nih.gov/health-information/diabetes/overview/what-is-diabetes#:˜:t=Diabetes %20is%20a%20disease%20that.to%20be%20used%20for%20 energy.

The present disclosure also relates generally to methods for treating obesity by administering a specific probiotic Lactobacillus johnsonii 456 to a mammal, such as an animal or patient or person in need. Obesity is a chronic condition or disease that affects millions of adults, adolescents, and children in the United States. Obesity results from a combination of genetic susceptibility, biological pathways related to metabolism and body weight regulation, and environmental factors. According to the World Health Organization, obesity has nearly tripled since 1975. In the U.S., 42% of adults were considered obese (2017-2018). Among major ethnic groups, only non-Hispanic Asian men (17.4%) and women (17.2%) had rates significantly lower than the average. Some 18.5% of children and adolescents in the U.S. (2015-2016), about 13.7 million, were obese. Childhood obesity is more common among Hispanics (25.8%) and non-Hispanic blacks (22%) than other population groups. The Centers for Disease Control and Prevention (CDC) compiles state-level estimates of obesity prevalence among U.S. adults.

Obesity is one of the signs of metabolic syndrome, along with high blood pressure, blood sugar, and cholesterol levels. People affected by obesity have a higher chance of developing serious health problems, including asthma, type 2 diabetes, high blood pressure, cardiovascular disease, stroke, and many cancers, which together are among the leading causes of preventable or premature death. Children with obesity may be at a greater risk for allergies.

According to the CDC, being overweight and/or obese are associated with at least 13 types of cancer. Several studies also show that patients affected by obesity have a higher chance of developing serious health problems, including immunological disorders, asthma, type 2 diabetes, high blood pressure, cardiovascular disease, stroke, and many cancers, which together are among the leading causes of preventable or premature death. https://www.niehs.nih.gov/health/topics/conditions/obesity/index.cfm

Certain intestinal microbiota are capable of modulating oxidative stress levels inside the body. However, not all probiotics survive the trip from mouth to the intestines, nor do they all have long-term survival within a patient's intestines.

In order to overcome the diseases described above, there are many medications sometimes with very detrimental side effects and no probiotic on the market has been shown to treat all of those diseases and conditions simultaneously. There is a need for a probiotic with minimal to no negative side effects capable of treating inflammatory diseases, cancer, cardiovascular diseases, diabetes and obesity, that survives the patient's stomach environment in order to reach the intestines, and persists within the intestines for a significant length of time.

The embodiments described below address the above-mentioned diseases and conditions which most importantly are some of the most lethal and commonly occurring in mammals, including both animal and human populations. Hereinafter, the term “patient” shall refer generally to mammals, including both animal and human populations.

### BRIEF SUMMARY

In accordance with one embodiment of the present disclosure, there is contemplated a method of treating diseases and conditions within a patient by administering Lactobacillus johnsonii 456.

In certain embodiments of the present disclosure, the L. johnsonii 456 is contained within any capsule or sleeve.

In certain embodiments of the present disclosure, the L. johnsonii 456 is contained within a food composition. More particularly, in some embodiments L. johnsonii 456 may be contained within yogurt, ice cream, and/or candy.

In certain embodiments of the present disclosure, the L. johnsonii 456 is contained within beverage. More particularly, in some embodiments L. johnsonii 456 may be contained within beer or kambucha.

In certain embodiments of the present disclosure, the L. johnsonii 456 is contained within a cosmetic cream.

The methods described herein involving the medical use of L. johnsonii may be used to achieve beneficial health outcomes in the treatment of various diseases or conditions within a mammal, such as an animal, patient or person (collectively referred to as “patient”).

The methods described herein may be used to treat inflammatory diseases within a patient.

The methods described herein may be used to treat pneumonia within a patient.

The methods described herein may be used to treat enteritis within a patient.

The methods described herein may be used to treat cancer within a patient.

The methods described herein may be used to treat colon cancer within a patient.

The methods described herein may be used to treat blood cancer within a patient.

The methods described herein may be used to treat cardiovascular disease within a patient.

The methods described herein may be used to treat myocardial infarction or heart attack within a patient.

The methods described herein may be used to reduce the glycemic index within a patient.

The methods described herein may be used to treat diabetes within a patient.

The methods described herein may be used to treat obesity within a patient.

The methods described herein shows that Lactobacillus johnsonii 456 may be used to treat the most prevalent and lethal diseases and conditions of a patient.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a table showing the results of a clinical trial involving the medical use of L. johnsonii 456 to treat inflammatory diseases, namely pneumonia and enteritis;

FIG. 2 is a table showing the results of a clinical trial involving the medical use of L. johnsonii 456 to treat cancers, namely lymphoid carcinoma and colon cancer; and

FIG. 3 is a table showing the results of a clinical trial involving the medical use of L. johnsonii 456 to treat cardiovascular disease, namely myocardial infarction or heart attack, as well as diabetes and obesity by reducing the glycemic index.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

The inventor of the present disclosure has been studying the impacts of the probiotic Lactobacillus johnsonii 456 on many diseases, including inflammatory diseases, cancers, cardiovascular disease, diabetes and obesity which are the statistically most prevalent and lethal diseases and conditions of mammals, such as animals and humans (collectively referred to as “patients”).

Inflammation is involved in most deadly diseases, such as, but not limited to, cancer, heart disease, neurodegenerative disease, inflammatory bowel disease, ulcerative colitis, lupus, Crohn's disease, celiac disease, arteriosclerosis, arthritis, fibrosis, asthma, diabetes, autism, aging, and the like. By administering L. johnsonii 456 to a patient, there can be achieved great results in the treatment of diseases.

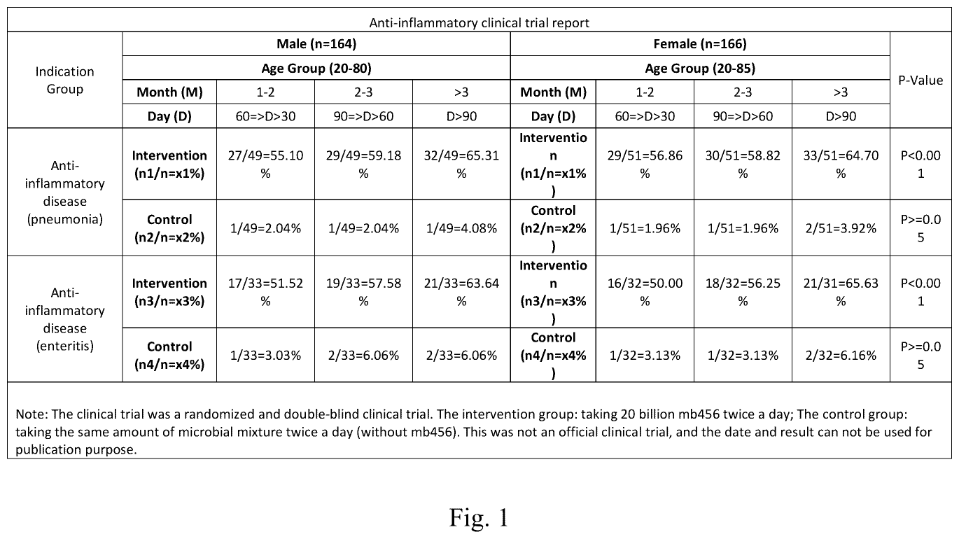

These beneficial results were shown in randomized, double-blind human clinical trials, as shown in the table of FIGS. 1-3. In particular, the intervention groups were given 20 billion CFUs of L. johnsonii 456 in probiotic capsules twice a day, whereas the control groups were given capsules lacking L. johnsonii 456 twice a day.

Study results showed vast improvements in the intervention groups with various types of the most prevalent and lethal diseases of patients. See FIG. 1-3.

Specifically, both male and female human patients given L johnsonii 456 who had inflammatory disease pneumonia showed a greater than 16× improvement after three months compared to the control group. See. FIG. 1.

According to the present invention, male and female human patients given L. johnsonii 456 who had inflammation symptomology from enteritis had rates of improvement greater than 10× after three months compared to the control group. These results are highly significant at a p-value smaller than 0.01. This is highly relevant because 50% of all people die from inflammation caused diseases. See FIG. 1.

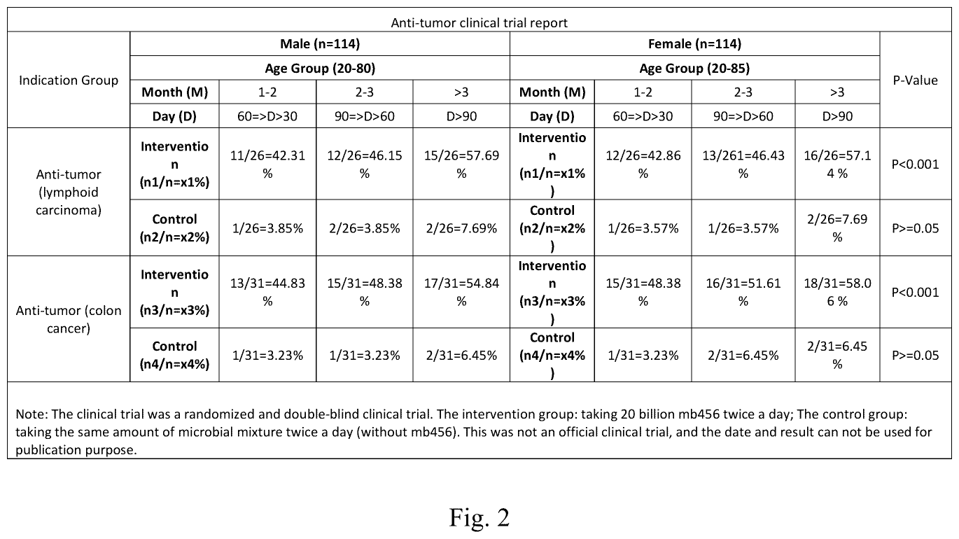

According to the present invention, study results also showed vast improvements in the intervention groups with various types of cancer. See FIG. 2. Also, male and female human patients given L. johnsonii 456 who had lymphoid carcinoma showed rates of improvement of approximately 7.5× after three months compared to that of the control group. See FIG. 2.

According to the present invention, male and female human patients given L. johnsonii 456 who had colon cancer had rates of improvement greater than 8.5× after three months compared to the control group. See FIG. 2. These results are highly significant at a p-value of smaller than 0.01. This is also highly relevant since 25% of all people have some form of cancer in their life time.

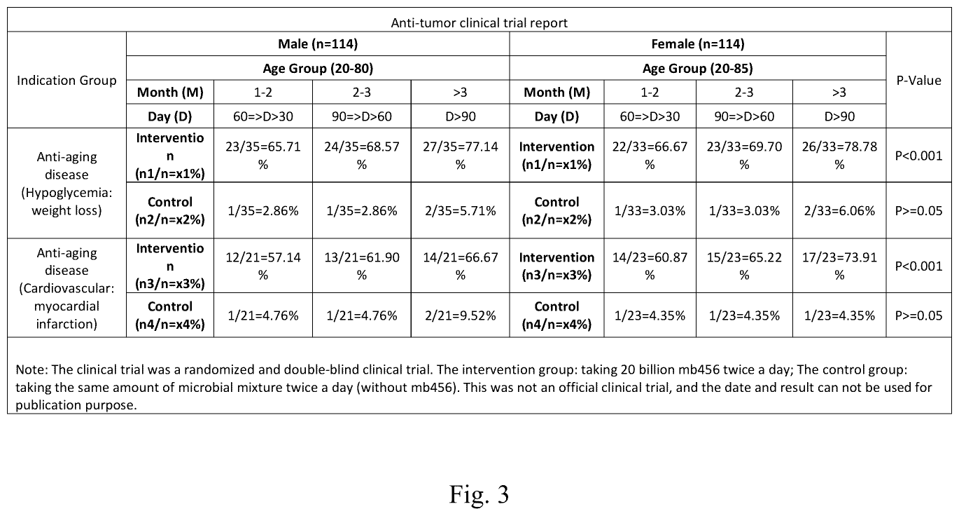

According to the present invention, male human patients given L. johnsonii 456 who had cardiovascular disease with myocardial infarction experienced rates of improvement approximately 7× better than the control group after three months, while female human patients with the heart disease myocardial infarction showed rates of improvement nearly 17× greater than the control group after three months. See FIG. 3. These results are highly significant at a p-value of smaller than 0.01. This is highly relevant since 25% of all people have some form of cardiovascular disease around the world in their life time, and even almost half of all adults in the United States having at least one form of cardiovascular disease.

According to the present invention, in yet a third study, male and female human patients given L. johnsonii 456 showed reduction of the glycemic index 13× after three months compared to the control group. See FIG. 3. These results are significant at a p-value smaller than 0.01. These results are highly relevant because the reduction of the glycemic index levels benefits all diabetics and all overweight patients with 50% of the population in the United States being overweight. The inventor himself lost 20 pounds using Lactobacillus johnsonii 456 every day.

As such, it can be seen that the described methods of treating inflammatory diseases, cancers, cardiovascular diseases, diabetes and obesity by administering L. johnsonii to patients has significant beneficial results and has been shown to be useful in treating various unrelated diseases which constitute the most prevalent and most lethal diseases and conditions of people. Further clinical trials are being conducted regarding the effect of L. johnsonii on other diseases in view of the trial results depicted in FIGS. 1-3. For instance, L. johnsonii relieves symptoms of depression in mice. Similar results are expected for the treatment of mammals including human patients for a host of other diseases including those listed below. https://dph.illinois.gov/topics-services/diseases-and-conditions/diseases-a-z-list.html

The above description is given by way of example, and not limitation. Given the above disclosure, one skilled in the art could devise variations that are within the scope and spirit of the invention disclosed herein, including the method of administering the L. johnsonii 456 to mammals such as patients and people Further, the various features of the embodiments disclosed herein can be used alone, or in varying combinations with each other such as comorbidities, like obese people may have higher incidences of all the other diseases treated here, and are not intended to be limited to the specific combination described herein. Thus, the scope of the claims is not to be limited by the illustrated embodiments.

What is claimed is:

1. A method of treating inflammatory disease, said method comprising administering L. johnsonii 456 to a patient with inflammatory disease.

2. The method of claim 1 wherein the inflammatory disease is enteritis.

3. The method of claim 1 wherein the inflammatory disease is pneumonia.

4. The method of claim 1 wherein the inflammatory disease is inflammable bowel disease.

5. The method of claim 1 wherein the inflammatory disease is asthma.

6. The method of claim 1, wherein the L. johnsonii 456 is contained within one of a food composition, capsule, sleeve or beverage.

7. A method of treating cancer, said method comprising administering L. johnsonii 456 to a patient with cancer.

8. The method of claim 7, wherein the L. johnsonii 456 is contained within one of a food composition, capsule, sleeve or beverage.

9. The method of claim 7 wherein the cancer is colon cancer.

10. The method of claim 7 wherein the cancer is small intestinal cancer.

11. The method of claim 7 wherein the cancer is a blood cancer.

12. The method of claim 11 wherein the blood cancer is lymphoid carcinoma.

13. The method of claim 11 wherein the blood cancer is leukemia.

14. The method of claim 11 wherein the blood cancer is myeloma.

15. A method of treating cardiovascular disease, said method comprising administering L. johnsonii 456 to a patient with cardiovascular disease.

16. The method of claim 15, wherein the L. johnsonii 456 is contained within one of a food composition, capsule, sleeve, beverage.

17. The method of claim 15 wherein the cardiovascular disease is myocardial infarction.

18. The method of claim 15 wherein the cardiovascular disease is ischemic stroke.

19. The method of claim 15 wherein the cardiovascular disease is congestive heart failure.

20. A method of reducing the glycemic index, said method comprising administering L. johnsonii 456 to a patient.

21. The method of claim 20, wherein the L. johnsonii 456 is contained within one of a food composition, capsule, sleeve, beverage.

22. A method of treating diabetes, said method comprising administering L. johnsonii 456 to a patient with diabetes.

23. The method of claim 22, wherein the L. johnsonii 456 is contained within one of a food composition, capsule, sleeve, beverage.

24. A method of treating obesity, said method comprising administering L. johnsonii 456 to a patient with obesity.

25. The method of claim 24, wherein the L. johnsonii 456 is contained within one of a food composition, capsule, sleeve, beverage.
